(19) United States
(12) Patent Application Publication
(10) Pub. No.: US 2026/0306522 A1
(43) Pub. Date: Oct. 8, 2026

(54) NUTRITIONAL COMPOSITION AND METHOD FOR PROMOTING SLEEP AND/OR FOR TREATING AND/OR PREVENTING SLEEP DISORDERS

(71) Applicant: SOCIÉTÉ DES PRODUITS NESTLÉ S.A., Vevey (CH)

(72) Inventor: Laurent FERRIER, Servion (CH)

(21) Appl. No.: 19/483,726

(22) PCT Filed: May 7, 2024
(86) PCT No.: PCT/EP2024/062523

§ 371 Date: Nov. 12, 2025

(30) Foreign Application Priority Data
May 16, 2023 (EP) 23173707.3

(51) Int. Cl. A61K 31/702; A23L 33/00

(52) U.S. Cl. CPC A61K 31/702; A23L 33/40

(57) ABSTRACT

The present invention relates to a combination of galacto-oligosaccharides (GOS) and a mix of human oligosaccharides (HMOs), or nutritional compositions comprising the combination, for use in promoting sleep and/or treating and/or preventing sleep disorders in a subject, wherein the mix consists of 2′-fucosyllactose (2′FL) and difucosyllactose (DFL) or lacto-N-tetraose (LNT) and 6′-sialyllactose (6′SL). The present invention also relates to the use of a combination of galacto-oligosaccharides (GOS) and a mix of human oligosaccharides (HMOs), or nutritional compositions comprising the combination, for use in promoting sleep and/or treating and/or preventing sleep disorders in a subject, wherein the mix consists of 2′-fucosyllactose (2′FL) and difucosyllactose (DFL) or lacto-N-tetraose (LNT) and 6′-sialyllactose (6′SL). Furthermore, the present invention relates to a method for in promoting sleep and/ or in treating and/or preventing sleep disorders in a subject, the method comprising administering to the subject a combination or a nutritional composition according to the invention.

### FIELD OF THE INVENTION

The present invention relates to a combination of galacto-oligosaccharides (GOS) and a mix of human oligosaccharides (HMOs), or nutritional compositions comprising the combination, for use in promoting sleep and/or treating and/or preventing sleep disorders in a subject, wherein the mix consists of 2′-fucosyllactose (2′FL) and difucosyllactose (DFL) or lacto-N-tetraose (LNT) and 6′-sialyllactose (6′SL).

The present invention also relates to the use of a combination of galacto-oligosaccharides (GOS) and a mix of human oligosaccharides (HMOs), or nutritional compositions comprising the combination, for use in promoting sleep and/or treating and/or preventing sleep disorders in a subject, wherein the mix consists of 2′-fucosyllactose (2′FL) and difucosyllactose (DFL) or lacto-N-tetraose (LNT) and 6′-sialyllactose (6′SL).

Furthermore, the present invention relates to a method for in promoting sleep and/or in treating and/or preventing sleep disorders in a subject, the method comprising administering to the subject a combination or a nutritional composition according to the invention.

### BACKGROUND OF THE INVENTION

Sleep disorders refers to medical conditions that affect sleep quality, timing or duration and impact a subject's ability to properly function while being awake. They are more than about 80 different types of sleep disorders and amongst those the most frequent are insomnia, poor sleep quality, early waking, circadian rhythm disorders, parasomnias, sleep-related movement disorders and sleep-related breathing disorders (SBDs). Sleep disorders can contribute to other medical problems such as obesity and physical problems that may interfere with everyday life. In healthy individuals, short-term consequences include a heightened stress response, pain depression, anxiety and cognition, memory and performance deficit. In adolescents and children, sleep disorders can lead to poor school performance and behavior problems (Medic G. et al. Nat Sci Sleep 2017; 9:151-161).

Melatonin (N-acetyl-5-methoxytryptamine) is an indoleamide hormone primarily secreted by the pineal gland and released into the bloodstream exclusively at night following the circadian rhythm. Melatonin acts as a circadian rhythm synchronizer and endogenous antioxidant; it is the main hormone involved in the control of sleep-wake cycle. Melatonin supplementation orally has been shown to be a safe and effective method to improve sleep onset latency, duration, and quality in children (Chang Y S et al. JAMA Pediatr. 2016; 170:35-42), in adolescents (Barlett D J et al. Med J Austr. 2013; 199: S16-S20), older adults (Lahteenmaki R et al. Br J Clin Pharmacol 2014; 77:975-985) and postmenopausal women (Amstrup A K et al. Nutr J. 2015; 14:102). However, the bioavailability of orally administered melatonin preparations in human subject is limited and variable (Lee B J et al. Int J. Pharm. 1995; 124:119-127). Melatonin absorption was shown to occur throughout the gastro-intestinal (GI) tract, with the greatest absorption being in the rectum and the ileum and the least in the stomach (Tran H T et al. Int J. Pharm. 2009; 378:9-16). The absorption of melatonin is affected by pharmaceutical excipients (Tran H T et al. Int J. Pharm. 2009; 378:9-16) and the most suitable dose ranges and pharmaceutical preparations for melatonin administration are yet to be clearly defined.

There is no nutritional solution shown or available to naturally increase the synthesis of melatonin by the microbiota of an individual, in particular to improve sleep quality and/or treat and/or prevent sleep disorders.

Accordingly, there is a need to provide nutritional solutions capable of improving sleep and/or treating and/or preventing sleep disorders in a subject, for example by increasing the the production of melatonin by the gut microbiota of the subject.

Any reference to prior art documents in this specification is not to be considered an admission that such prior art is widely known or forms part of the common general knowledge in the field. As used in this specification, the words “comprises”, “comprising”, and similar words, are not to be interpreted in an exclusive or exhaustive sense. In other words, they are intended to mean “including, but not limited to”.

### SUMMARY OF THE INVENTION

The present inventors have surprisingly found that a combination of galacto-oligosaccharides (GOS) and a mix of human milk oligosaccharides (HMOs) can advantageously be used to improve/promote sleep and/or treat and/or prevent sleep disorders in a subject because the combination showed increased biosynthesis of melatonin by the gut microbiota of the subject. Although some evidences show gastrointestinal tract may be a source of extrapineal melatonin, probably produced in the serotonin-rich enterochromaffin cells of the GI mucosa (Bubenik G A Biol Signals Recept 2001; 10(6): 350-66), biosynthesis of melatonin by the gut microbiota was not previously known. The melatonin is not supplemented orally, thereby avoiding the problem of accurately defining the appropriate dosage. Furthermore, the melatonin synthesized by the gut microbiota may be efficiently delivered and absorbed throughout the gastro-intestinal tract (Tran H T et al. Int J Pharm 2009; 378(1-2): 6-16) and thereby act on sleep onset, latency, duration, and quality. Thus, the invention provides a way to increase the production of a metabolite (i.e. melatonin) with known benefits on sleep. The mix of HMOs consists of either 2′-FL and DFL or of LNT and 6′-SL. The impact of a combination of GOS and a mix of HMOs on the biosynthesis of melatonin was not previously known.

In the experiment at the basis of the present invention, a combination of GOS and either 2′FL and DFL or LNT and 6′-SL resulted in increased biosynthesis of melatonin by gut microbiota. Thus, the experiment indicates the role played by the combination in promoting the biosynthesis of melatonin.

An object of the present invention is to improve the state of the art and to provide a new combination or nutritional composition, or at least to provide a useful alternative, to overcome at least some inconveniences described above.

The object of the present invention is achieved by the subject matter of the independent claims. The dependent claims further develop the idea of the present invention.

Thus, in a first aspect, the present invention provides a combination of galactooligosaccharides (GOS) and a mix of human milk oligosaccharides (HMOs) for use in promoting sleep and/or in treating and/or preventing sleep disorders in a subject, wherein the mix of HMOs consists of 2′-fucosyllactose (2′FL) and difucosyllactose (DFL) or lacto-N-tetraose (LNT) and 6′-sialyllactose (6′SL).

In another aspect, the present invention provides a nutritional composition comprising a combination of galacto-oligosaccharides (GOS) and a mix of human milk oligosaccharides (HMOs) for use in promoting sleep and/or in treating and/or preventing sleep disorders in a subject, wherein the mix of HMOs consist of 2′-fucosyllactose (2′FL) and difucosyllactose (DFL) or lacto-N-tetraose (LNT) and 6′-sialyllactose (6′SL).

In a further aspect, the present invention provides a method for promoting sleep and/or treating and/or preventing sleep disorders in a subject, the method comprising administering to the subject a combination or a nutritional composition according to the invention.

In another aspect, the invention provides the use of a combination according to the invention or of a nutritional composition according to the invention for increasing the production of melatonin by the gut microbiota of a subject.

In some embodiments, the use or the method according to the invention is non-therapeutic. Suitably, the use or the method is for increasing the production of melatonin by the gut of a subject; for treating and/or preventing insomnia, poor sleep quality, early waking, circadian rhythm disorders, parasomnias, sleep-related movement disorders and sleep-related breathing disorders (SBDs) in a subject.

In some embodiments of the methods and uses of the invention, the combination or nutritional composition according to the invention is used for the promotion of sleep, for example for improving sleep onset latency, sleep quality and sleep quality.

In some embodiments of the methods and uses of the invention, the combination or nutritional composition according to the invention is used for the treatment of sleep disorders, for example insomnia, poor sleep quality, early waking, circadian rhythm disorders, parasomnias, sleep-related movement disorders and sleep-related breathing disorders (SBDs).

In some embodiments of the methods and uses of the invention, the combination or nutritional composition according to the invention is used for the treatment and/or prevention of sleep disorders.

In some embodiments of the methods and uses of the invention, the combination or nutritional composition according to the invention is used for the treatment of insomnia, poor sleep quality, early waking, circadian rhythm disorders, parasomnias, sleep-related movement disorders and sleep-related breathing disorders (SBDs).

In some embodiments of the methods and uses of the invention, the combination or nutritional composition according to the invention is used for the prevention of sleep disorders, for example insomnia, poor sleep quality, early waking, circadian rhythm disorders, parasomnias, sleep-related movement disorders and sleep-related breathing disorders (SBDs).

### BRIEF DESCRIPTION OF THE DRAWINGS

FIG. 1—R elative abundance of melatonin, expressed as peak area, after 24 h of fermentation by toddler's microbiota of digested milk matrix supplemented by GOS, 2′FL/DFL, LNT/6′SL, GOS+2′FL/DFL or GOS+LNT/6′SL. Dots represent individual values, line is at the median, box are 25/75 percentiles and whiskers at min/max range. Different letters denote significant differences.

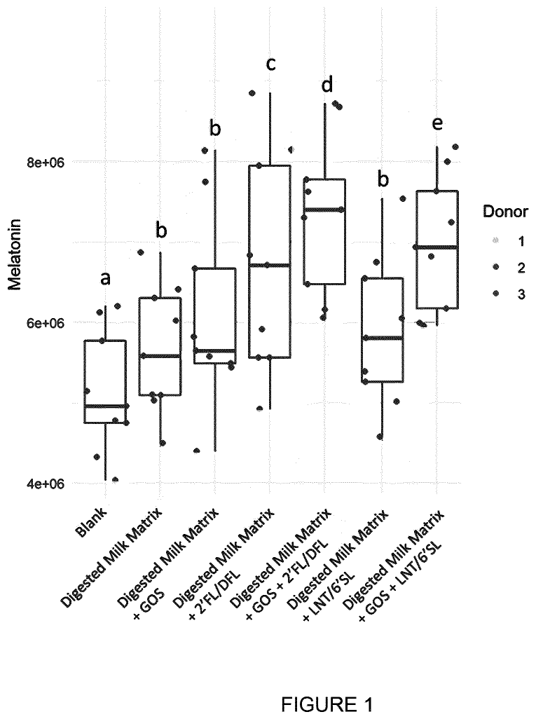

FIG. 2—R elative abundance of melatonin, expressed as the base-2 logarithm of the ratio between the value of melatonin after 24 h of fermentation by toddler's microbiota of digested milk matrix supplemented by GOS, 2′FL/DFL, LNT/6′SL, GOS+2′FL/DFL or GOS+LNT/6′SL, and the value of melatonin obtained after fermentation of the digested milk matrix alone. Dots represent individual values, line is at the median, box are 25/75 percentiles and whiskers at min/max range. Different letters denote significant differences.

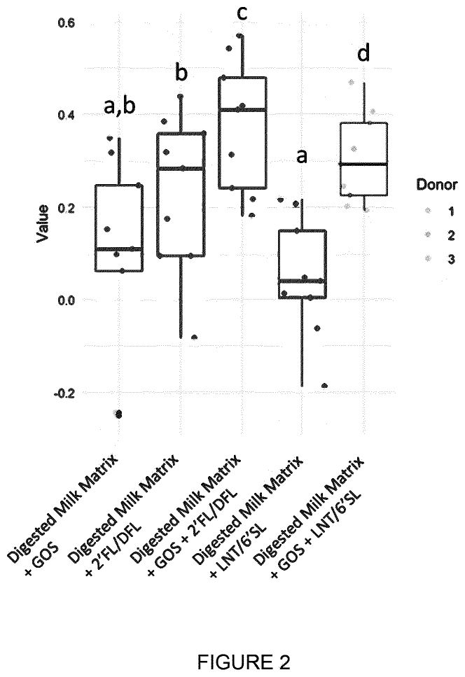

### DETAILED DESCRIPTION OF THE INVENTION

### Definitions

Various preferred features and embodiments of the present invention will now be described by way of non-limiting examples. The skilled person will understand that they can combine all features of the invention disclosed herein without departing from the scope of the invention as disclosed.

Any reference to prior art documents in this specification is not to be considered an admission that such prior art is widely known or forms part of the common general knowledge in the field. All publications mentioned in the specification are herein incorporated by reference.

In the context of the invention, the terms “comprising” or “comprises” do not exclude other possible elements. The composition of the present invention, including the many embodiments described herein, can comprise, consist of, or consist essentially of the essential elements and limitations of the invention described herein, as well as any additional or optional ingredients, components, or limitations described herein or otherwise depending on the needs. The practice of the present invention will employ, unless otherwise indicated, conventional techniques which are within the capabilities of one of ordinary skill in the art. Such techniques are explained in the literature. Unless defined otherwise, all technical and scientific terms used herein have the same meaning as commonly understood by one of ordinary skill in the art.

Numeric ranges are inclusive of the numbers defining the range and all percentages disclosed herein are on a w/w basis, unless stated otherwise. As used herein the term “about” means approximately, in the region of, roughly, or around. When the term “about” is used in conjunction with a numerical value or range, it modifies that value or range by extending the boundaries above and below the numerical value(s) set forth. In general, the terms “about” and “approximately” are used herein to modify a numerical value(s) above and below the stated value(s) by 10%.

The term “subject” as used therein refers to a mammal. Mammal includes, but is not limited to, rodents, aquatic mammals, domestic animals such as dogs and cats, farm animals such as sheep, pigs, cows and horses, and humans. In one embodiment, the mammal may be a cat, a dog or a human. The human may be an adult, for example a man or a woman, an adolescent, an infant, a toddler or a young child. In one embodiment of the invention, the subject is a mammal selected from the group consisting of a cat, a dog and a human.

The term “infant” means a child under the age of 12 months. The term infant includes both infants born at term and infant born preterm. The expression “young child” means a child aged between one and three years, also called toddler. The expression “child” means a child between one and ten years of age, including toddler and pre-school children. Preferably, the expression “child” means a child between three and five years of age.

The term adolescent means an individual aged between 10 and 19 years of age.

The terms “supporting”, “promoting”, “enhancing” or “improving” may be used interchangeably in the context of the present invention. They should be understood as comprising supporting or helping sleep, for example by promoting sleep quality, decreasing difficulty falling asleep, and reducing sleep disorders. The subject may not suffer from a disease but may be susceptible to the development of unhealthy conditions, such as acute fatigue, depression or irritability that can interfere with everyday life.

The terms “treat” or “treating” mean to address a medical condition or disease with the objective of improving or stabilizing an outcome in the person being treated or addressing an underlying nutritional need. Treating, therefore, includes the dietary or nutritional management of the medical condition or disease by addressing nutritional needs of the person being treated. Treating includes the elimination, reduction or amelioration of symptoms associated with the medical condition or disease.

The terms “prevent” or “preventing” mean to diminish the risk of onset or recurrence of a medical condition or disease. Both primary and secondary prevention are thus contemplated.

Within the context of the present invention, the expression “sleep disorders” refers to conditions that affect the sleep quality, timing or duration and impact a person's ability to properly function when awake. Sleep disorders are characterized by trouble falling or remaining asleep, difficulty to stay awake during the day, imbalances in the circadian rhythm.

The term “insomnia” refers to persistent difficulty to fall asleep (sleep initiation), sleep consolidation and staying asleep. hm that interfere with a healthy sleep schedule or unusual behaviors that disrupt sleep. Individuals suffering from chronic insomnia show an increased risk for developing psychiatric conditions, such as depression or anxiety, and can have intense response to stressful events. Melatonin administration is efficient for treating insomnia.

Within the context of the present invention, the expression “poor sleep quality” refers to a sleep that is not restful and restorative.

Within the context of the present invention, the expression, the expression “sleep latency” refers to the measurement of how long it takes to an individual to fall asleep. It is usually accepted that falling asleep within 30 minutes or less after the time the individual goes to bed is the sign of a good sleep quality.

Within the context of the present invention, the expression “sleep awaking” measures how often an individual is wake up during the night. Frequent wakefulness at night can disrupt sleep cycle and reduce sleep quality. Waking up once or not at all usually suggests a good sleep quality.

Within the context of the present invention, the term “wakefulness” measures how many minutes spent awake by an individual during the night after bedtime. 20 minutes or less of wakefulness during the night is usually a sign of good sleep quality.

Within the context of the present invention, the expression “sleep efficiency” measure to the time spends actually sleeping while in bed. A measurement of 85 percent is usually suggesting a good sleep quality.

Within the context of the present invention, the expression “circadian rhythm disorders” refers to a group of disorders that are related to disturbance in the sleep-wake cycle due to problems with the circadian rhythm. The circadian rhythm is a cycle of approximatively 24 hours that is naturally present in all living organisms and regulates the back-and-forth between states of rest and activity. In humans, this rhythm is controlled by the internal clock located in hypothalamus. Melatonin plays a role in the regulation of the circadian rhythm, attenuating the wake-promoting signal of the circadian clock thus promoting sleep (Liu C et al. Neuron 1997; 19:91-102) and is used as treatment of circadian rhythm disorders and sleep disorders.

Within the context of the present invention, the term “parasomnias” refers to undesirable events that occur during NREM (non-rapid eye movement) or REM (rapid eye movement) sleep or transitions to and from sleep. Melatonin has beneficial effects in patients suffering of parasomnias (McGrane A R et al. Sleep Med. 2015; 16(1): 19-26).

The term “narcolepsy” refers to a disorder of circadian rhythm and REM sleep deficiency, that makes the individual feeling excessively tired during the day despite getting an adequate amount of sleep. Melatonin alters sleep architecture in narcolepsy (Xie Z et al. Neurological Research 2017; 39:559-565) and may help regulating and inducing sleep, increasing REM sleep time and improving sleep quality, thereby reducing narcolepsy.

Within the context of the present invention, the expression “sleep-related movement disorders” refers to sleep disorders in which repetitive movements interfere with sleep. Sleep-related movements disorders include: Restless Leg Syndrome (RLS), a condition in which the individual experience strong, sometimes irresistible urges to move their limbs, Periodic Limb Movement Disorders (PLMD) that involves repetitive movements of the arms, legs or feet during sleep, Sleep-related bruxism in which an individual clenches or grinds their teeth during sleep, sleep-related leg cramps in which the individual experience sudden and unvoluntary muscle spasm and sleep-related rhythmic movement disorder which is a condition characterized by repetitive, rhythmic movements, usually body rocking, headbanging or head rolling, occurring during sleep. Melatonin may have a beneficial effect to reduce sleep-related movement disorders (Metin O et al. Sleep Sci. 2019; 12(1) 53-56).

Within the context of the present invention, the expression “sleep-related breathing disorders (SBDs)” refers to abnormal and difficult respiration during sleep, including chronic snoring and sleep apnea. Melatonin has been shown to improve the complications caused by SBDs (Xie Z. et al. Nerological Research 2017; 39:559-565).

The expression “nutritional composition” means a composition which nourishes a subject. This nutritional composition is usually to be taken orally or parenterally, and it usually includes a lipid or fat source and a protein source. A carbohydrate source may also be included. Furthermore, the nutritional composition may comprise the following micronutrients: vitamins, minerals, fiber, phytochemicals, antioxidants, prebiotics, probiotics, bioactives, metabolites (e.g. butyrate, Docosahexaenoic acid (DHA), Eicosapentaenoic acid (EPA), Gamma-Linolenic acid (GLA)) and any combination thereof. The composition may also contain food additives such as stabilizers (when provided in liquid or solid form) or emulsifiers (when provided in liquid form). The nutritional composition may be in solid (e.g. powder), semi-solid or liquid form; it may for example be provided in the form of a pill, a tablet, a lozenge, a chewy capsule or tablet, a tablet or capsule, or a powder supplement that can for example be dissolved in water or sprinkled on food. In one embodiment, the nutritional composition of the invention is a synthetic nutritional composition.

In a particular embodiment, the combination or composition of the present invention is a “synthetic combination” or “synthetic nutritional composition”. The expression “synthetic combination” or “synthetic nutritional composition” means a mixture obtained by chemical and/or biological means, which can be chemically identical to the mixture naturally occurring in mammalian milks (i.e. the synthetic combination or synthetic composition is not breast milk).

The combination or nutritional composition of the present invention can be a food product, a functional food product, a healthy aging food product, a dairy product, a dairy alternative product, a milk or milk-based product, a beverage, a diet product, a petfood product, an infant formula, a follow-up formula or a follow-on formula, a baby food, an infant cereal composition, growing-up milk (or GUM) or a fortifier. In one embodiment, the combination or nutritional composition of the present invention is an infant formula, a follow-up formula or a follow-on formula, a baby food, an infant cereal composition, growing-up milk (or GUM) or a fortifier.

The term “food product,” as used herein, refers to any kind of product that may be safely consumed by a human or an animal. A food product may be in solid, semi-solid or liquid form and may comprise one or more nutrients, foods or nutritional supplements. For instance, the food product may additionally comprise the following nutrients and micronutrients: a source of proteins, a source of lipids, a source of carbohydrates, vitamins and minerals. The food product may also contain antioxidants, stabilizers (when provided in solid form) or emulsifiers (when provided in liquid form).

The term “functional food product,” as used herein, refers to a food product providing an additional health-promoting or disease-preventing function to the individual.

The term “healthy ageing product,” as used herein, refers to a product providing an additional health-promoting or disease-preventing function related to healthy ageing to the individual.

The term “dairy products,” as used herein, refers to food products produced from milk or fractions of milk from animals such as cows, goats, sheep, yaks, horses, camels, and other mammals. Examples of dairy products are low fat milk (e.g., 0.1%, 0.5% or 1.5% fat), fat-free milk, milk powder, whole milk, whole milk products, butter, buttermilk, buttermilk products, skim milk, skim milk products, high milk-fat products, condensed milk, creme fraiche, cheese, ice cream and confectionery products, probiotic drinks or probiotic yoghurt type drinks.

The term “dairy alternative product,” as used herein, refers to products similar to dairy products but produced without milk.

The term “milk,” as used herein, is defined by Codex Alimentarius as the normal mammary secretion of milking animals obtained from one or more milkings without either addition to it or extraction from it, intended for consumption as liquid milk or for further processing.

The term “beverage product,” as used herein, refers to a nutritional product in liquid or semi-liquid form that may be safely consumed by an individual.

The term “diet product,” as used herein, refers to a food product with a restricted and/or reduced caloric content.

The term “pet food product,” as used herein, refers to a nutritional product that is intended for consumption by pets. A pet, or companion animal, as referenced herein, is to be understood as an animal selected from dogs, cats, birds, fish, rodents such as mice, rats.

The expression “infant formula” as used herein refers to a foodstuff intended for particular nutritional use by infants during the first months of life and satisfying by itself the nutritional requirements of this category of person (Article 2(c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The expression “infant formula” encompasses both “starter infant formula” and “follow-up formula” or “follow-on formula”.

A “follow-up formula” or “follow-on formula” is given from the 6th month onwards. It constitutes the principal liquid element in the progressively diversified diet of this category of person.

The expression “baby food” means a foodstuff intended for particular nutritional use by infants or young children during the first years of life.

The expression “infant cereal composition” means a foodstuff intended for particular nutritional use by infants or young children during the first years of life.

The expression “growing-up milk” (or GUM) refers to a milk-based drink generally with added vitamins and minerals, that is intended for young children or children.

The terms “fortifier” refers to liquid or solid nutritional compositions suitable for fortifying or mixing with human milk, infant formula, growing-up milk or human breast milk fortified with other nutrients. Accordingly, the fortifier can be administered after dissolution in human breast milk, in infant formula, in growing-up milk or in human breast milk fortified with other nutrients or otherwise it can be administered as a stand-alone composition. When administered as a stand-alone composition, the milk fortifier can be also identified as being a “supplement”.

An “oligosaccharide” is a saccharide polymer containing a small number (typically three to ten) of simple sugars (monosaccharides).

The term “galacto-oligosaccharides” refers to a type of non-digestible fiber with prebiotic activity. GOS are formed via enzymatic conversion of lactose. GOS generally comprise a chain of galactose units that arise through consecutive transgalactosylation reactions, with a terminal glucose unit, although a terminal galactose unit may be present instead. The degree of polymerization of GOS typically ranges from 2 to 8 monomeric units.

The term “HMO” or “HMOs” refers to human milk oligosaccharide(s). These carbohydrates are highly resistant to enzymatic hydrolysis, indicating that they may display essential functions not directly related to their caloric value. It has especially been illustrated that they play a vital role in the early development of infants and young children, such as the maturation of the immune system. Many different kinds of HMOs are found in the human milk. Each individual oligosaccharide is based on a combination of glucose, galactose, sialic acid (N-acetylneuraminic acid), fucose and/or N-acetylglucosamine with many and varied linkages between them, thus accounting for the enormous number of different oligosaccharides in human milk—over 130 such structures have been identified so far. Almost all of them have a lactose moiety at their reducing end while sialic acid and/or fucose (when present) occupy terminal positions at the non-reducing ends. The HMOs can be acidic (e.g. charged sialic acid containing oligosaccharide) or neutral (e.g. fucosylated oligosaccharide).

A “fucosylated oligosaccharide” is an oligosaccharide having a fucose residue. It has a neutral nature. Some examples are 2′FL (2′-fucosyllactose), 3-FL (3-fucosyllactose), difucosyllactose (DFL), Lacto-difucotetraose (LDFT)), lacto-N-fucopentaose (e.g. lacto-N-fucopentaose I, lacto-N-fucopentaose II, lacto-N-fucopentaose III, lacto-N-fucopentaose V), lacto-N-fucohexaose, lacto-N-difucohexaose I, fucosyllacto-N-hexaose, fucosyllacto-N-neohexaose, difucosyllacto-N-hexaose I, difucosyllacto-N-neohexaose II and any combination thereof.

The expression “N-acetylated oligosaccharide(s)” encompasses both “N-acetyl-lactosamine” and “oligosaccharide(s) containing N-acetyl-lactosamine”. They are neutral oligosaccharides having an N-acetyl-lactosamine residue. Suitable examples are LNT (lacto-N-tetraose), para-lacto-N-neohexaose (para-LNnH), LNnT (lacto-N-neotetraose) and any combinations thereof. Other examples are lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

A “precursor of HMO” is a key compound that intervenes in the manufacture of HMO, such as sialic acid and/or fucose.

A “sialylated oligosaccharide” is a charged sialic acid containing oligosaccharide, i.e. an oligosaccharide having a sialic acid residue. It has an acidic nature. Some examples are 3-SL (3′ sialyllactose) and 6′SL (6′ sialyllactose).

The combination or nutritional composition of the present invention can be in solid form (e.g. powder) or in liquid form. The amount of the various ingredients (e.g. the oligosaccharides) can be expressed in g/100 g of composition on a dry weight basis when it is in a solid form, e.g. a powder, or as a concentration in g/L of the composition when it refers to a liquid form (this latter also encompasses liquid composition that may be obtained from a powder after reconstitution in a liquid such as milk, water . . . , e.g. a reconstituted infant formula or a follow-on/follow-up formula or a growing-up milk or an infant cereal product or any other formulation designed for infant nutrition).

The term “prebiotic” means non-digestible carbohydrates that beneficially affect the host by selectively stimulating the growth and/or the activity of healthy bacteria such as bifidobacteria in the colon of humans (Gibson G R, Roberfroid M B. Dietary modulation of the human colonic microbiota: introducing the concept of prebiotics. J Nutr. 1995; 125:1401-12).

### Use

In one aspect the present invention provides a combination of galacto-oligosaccharides (GOS) and a mix of human milk oligosaccharides (HMOs) for use in promoting sleep and/or in treating and/or preventing sleep disorders in a subject, wherein the mix of HMOs consists of 2′-fucosyllactose (2′FL) and difucosyllactose (DFL) or lacto-N-tetraose (LNT) and 6′-sialyllactose (6′SL).

The combination may be in the form of a nutritional composition.

Accordingly, in another aspect, the present invention provides a nutritional composition comprising a combination of galacto-oligosaccharides (GOS) and a mix of human milk oligosaccharides (HMOs) for use in promoting sleep and/or in treating and/or preventing sleep disorders in a subject, wherein the mix of HMOs consist of 2′-fucosyllactose (2′FL) and difucosyllactose (DFL) or lacto-N-tetraose (LNT) and 6′-sialyllactose (6′SL).

There is preclinical and clinical evidence of the beneficial effect of exogenous melatonin in promoting sleep (Dawson D et al. J Pineal Res. 1993; 15(1): 1-12) and improving sleep onset latency, duration, and quality in children (Chang Y S et al. JAMA Pediatr. 2016; 170:35-42), in adolescents (Barlett D J et al. Med J Austr. 2013 ; 199: S16-S20), older adults (Lahteenmaki R et al. Br J Clin Pharmacol 2014; 77:975-985) and postmenopausal women (Amstrup A K et al. Nutr J. 2015; 14:102). Further evidence shows the effect of melatonin in treating sleep related disorders (Xie Z et al. Neurological research 2017; 39:559-565). However, no nutritional solution has previously been shown to naturally increase the synthesis of melatonin by the gut microbiota of a subject exposed to the nutritional solution.

Suitably, the combination or nutritional composition according to the present invention promote sleep and/or treat and/or prevent sleep disorders in a subject by increasing the production of melatonin by the gut microbiota of the subject. Some evidence suggests a relationship between circadian rhythms and the gut microbiome (Matenchuk B A et al. Sleep Med Rev 2020; 53:101340). Melatonin absorption was shown to occur throughout the gastro-intestinal (GI) tract (Tran H T et al. Int J. Pharm. 2009; 378(1-2): 9-16).

The inventors have found that a combination of GOS and either 2′FL and DFL or LNT and 6′-SL resulted in increased biosynthesis of melatonin by gut microbiota. The advantage of the present invention is that melatonin is not provided exogenously, for example in the form of oral supplementation, avoiding issues related to definition of correct dosage or modulation of absorption, for example due to excipients.

In some embodiments, the combination according to the invention or the nutritional composition according to the invention do not contain melatonin.

The inventors have also found that a mix of 2′FL and DFL induced biosynthesis of melatonin by the gut microbiota. Accordingly, in another aspect, the present invention provides a nutritional composition for use in promoting sleep and/or in treating and/or preventing sleep disorders in a subject, wherein the nutritional composition comprises a mix of HMOs consisting of 2′-fucosyllactose (2′FL) and difucosyllactose (DFL).

In some embodiments, promoting sleep improves the sleep onset latency, duration, and quality.

In some embodiments, treating or preventing sleep disorders improves the sleep quality and regulates the circadian rhythm in a subject. Thereby, the sleep related disorders are reduced. In another embodiment, treating or preventing sleep disorders reduces insomnia, poor sleep quality, sleep awaking, wakefulness, circadian rhythm disorders, parasomnias, narcolepsy, sleep-related movement disorders and sleep-related breathing disorders (SBDs).

In another aspect, the present invention provides a combination of galacto-oligosaccharides (GOS) and a mix of human milk oligosaccharides (HMOs) for use in increasing production of melatonin by gut microbiota in a subject, wherein the mix of HOMs consists of of 2′-fucosyllactose (2′FL) and difucosyllactose (DFL) or lacto-N-tetraose (LNT) and 6′-sialyllactose (6′SL).

In another aspect, the present invention provides a nutritional composition comprising a combination of galacto-oligosaccharides (GOS) and a mix of human milk oligosaccharides (HMOs) for use in increasing production of melatonin by gut microbiota in a subject, wherein the mix of HOMs consists of of 2′-fucosyllactose (2′FL) and difucosyllactose (DFL) or lacto-N-tetraose (LNT) and 6′-sialyllactose (6′SL).

In yet another aspect, the present invention provides a nutritional composition comprising a mix of human milk oligosaccharides (HMOs) for use in increasing production of melatonin by gut microbiota in a subject, wherein the mix of HOMs consists of of 2′-fucosyllactose (2′FL) and difucosyllactose (DFL).

Suitably, the biosynthesis of melatonin by the gut microbiota of the subject increased from 1 to 10-fold, such as by 1.1 to 2-fold, preferably by 1.1-fold to 1.5-fold. Suitably, the biosynthesis of melatonin by the gut microbiota of the subject is increased by at least 1-fold, such as by 1.1-fold, 1.3-fold, 1.4-fold, 1.5-fold or 2-fold.

Suitably, the base-2 logarithm of the ratio between the value of melatonin for the combination or composition according to the invention and the value of melatonin for a corresponding milk control is increased by from 0.1 to 1, suitably from 0.1 to 0.5.

The amount of melatonin secreted (i.e. melatonin biosynthesis) by the gut microbiota of the subject may be measured by methods known in the art. For example, the method disclosed herein may be used (see Example). Suitably, the amount of melatonin in fresh fecal samples exposed to the combination or nutritional composition according to the invention may be compared to samples not exposed to a combination or a nutritional composition according to the invention.

The gut-brain axis consists of bidirectional communication between the central nervous system (CNS) and the enteric nervous system (ENS) with the peripheral intestinal functions. The autonomic nervous system, hypothalamic-pituitary-adrenal (HPA) axis, and nerves within the gastrointestinal (GI) tract, all link the gut and the brain, allowing the brain to influence intestinal activities. The gut-brain axis may influence mood, cognitive function, stress response, memory, brain aging, intestinal system and sleep. Recent advances in research have described the importance of gut microbiota in influencing these interactions. This interaction between microbiota and gut-brain axis appears to be bidirectional, namely involving signaling from gut-microbiota to brain and from brain to gut-microbiota by means of neural, endocrine, immune, and humoral links (e.g. by means of the vagus nerve). In clinical practice, evidence of microbiota-GBA interactions comes from the association of dysbiosis with central nervous disorders (i.e. autism, anxiety-depressive behaviors) and functional gastrointestinal disorders (Wiley N C et al. Mod Trends Psychiatry 2021; 32:74-99).

In addition to the direct regulation of sleep by the brain, signals of peripheral organs have been shown to also play a role in sleep regulation. For example, the hypothalamic-pituitary-adrenal (HPA) axis is an endocrine pathway plays a critical role in sleep regulation (Buckley T M et al. J Clin Endocrinol Metab 2005; 90:3106-3114). Recently, a relationship between sleep disorders and changes in gut microbiota have been reported. The gut microbiota may use the gut-brain axis as a bridge to establish a connection with sleep, mainly including immune pathways, neural pathways and endocrine pathways.

Accordingly, in some embodiments, the combination or nutritional composition according to the invention participates to the communication between the gut and the brain by promoting secretion of neuroactive molecules.

In another aspect, the invention provides a combination of galacto-oligosaccharides (GOS) and a mix of human milk oligosaccharides (HMOs) for use in maintaining or promoting the communication between the gut and the brain by promoting secretion of neuroactive molecules in a subject, wherein the mix of HMOs consists of 2′-fucosyllactose (2′FL) and difucosyllactose (DFL) or of lacto-N-tetraose (LNT) and 6′ sialyllactose (6′SL).

In another aspect, the invention provides a nutritional composition comprising a combination of galacto-oligosaccharides (GOS) and a mix of human milk oligosaccharides (HMOs) for use in maintaining or promoting the communication between the gut and the brain by promoting secretion of neuroactive molecules in a subject, wherein the mix of HMOs consists of 2′-fucosyllactose (2′FL) and difucosyllactose (DFL) or of lacto-N-tetraose (LNT) and 6′ sialyllactose (6′SL).

In another aspect, the invention provides a nutritional composition comprising a mix of human milk oligosaccharides (HMOs) for use in maintaining or promoting the communication between the gut and the brain by promoting secretion of neuroactive molecules in a subject, wherein the mix of HMOs consists of 2′-fucosyllactose (2′FL) and difucosyllactose (DFL).

In a further aspect, the present invention provides a method for promoting sleep and/or for treating and/or preventing sleep disorders in a subject, the method comprising administering to the subject a combination according to the invention or a nutritional composition according to the invention.

In a further aspect, the present invention provides a method for maintaining or restoring the functionality of the bi-directional transmission pathways in the gut-brain axis to healthy levels in a subject, the method comprising administering to the subject a combination according to the invention or a nutritional composition according to the invention.

In a further aspect, the present invention provides a method for increasing production of melatonin by the gut of a subject, the method comprising administering the method comprising administering to the subject a combination according to the invention or a nutritional composition according to the invention.

In some embodiments of the method and uses of the invention, the combination or nutritional composition according to the invention is administered to a subject in an effective amount. The expression an “effective amount” means an amount that prevents a deficiency, treats a disease or medical condition in an individual or, more generally, reduces symptoms, manages progression of the diseases or provides a nutritional, physiological, or medical benefit to the individual.

In a further aspect, the present invention provides the use of a combination according to the invention or a nutritional composition according to the invention for promoting sleep and/or in treating and/or preventing sleep disorders in a subject. Suitably, the combination promotes sleep and/or treats and/or prevents sleep disorders in a subject by increasing production of melatonin by the gut microbiota of the subject.

In another aspect, the invention provides the use of a combination according to the invention or a nutritional composition according to the invention for maintaining or restoring the functionality of the bi-directional transmission pathways in the gut-brain axis to healthy levels in subject.

In another aspect, the invention provides the use of a combination according to the invention or a nutritional composition according to the invention for increasing production of melatonin by the gut microbiota of a subject.

In some embodiments, the use or method according to the invention is non-therapeutic. Suitably, the use or method is for increasing production of melatonin by the gut microbiota of a subject; for promoting sleep onset, latency in a subject; for promoting sleep duration in a subject; or for promoting sleep quality in a subject.

In some embodiments of the methods and uses of the invention, the combination or composition according to the invention is used for the treatment of sleep disorders in a subject.

In some embodiments of the methods and uses of the invention, the combination or composition according to the invention is used for the prevention of sleep disorders in a subject.

In some embodiments of the methods and uses of the invention, the sleep disorders treated and/or prevented consists of insomnia, poor sleep quality, sleep awaking, wakefulness, circadian rhythm disorders, parasomnias, narcolepsy, sleep-related movement disorders and sleep-related breathing disorders (SBDs).

### Target Population

In some embodiments of the methods and uses of the invention, the combination or composition is administered to an adult subject, for example a man or a woman, an adolescent, an infant, a young child and/or a child.

In one embodiment of the present invention, the combination or composition is administered to an infant, a young child or a child.

In one embodiment of the present invention, the combination or composition is administered to an infant.

In one embodiment of the present invention, the combination or composition is administered to a young child.

In one embodiment of the present invention, the combination or composition is administered to a child.

In one embodiment of the present invention, the infant or young children have an age ranging from 0 months to 36 months.

In another embodiment, the infant or young children have an age ranging from 0 months to 24 months, for example 18 months.

In a further embodiment, the infant has an age ranging from 0 months to 12 months, for example 9 or 6 months.

In some embodiments, the young child has an age ranging from one year to three years, for example 2 years.

In some embodiments, the child has an age ranging from three years to seven years. In some preferred embodiments, the child has an age ranging from three years to five years, for example four years.

The nutritional composition according to the invention is for use in adult subjects, for example a man or a woman, adolescents, infants, young children and/or children. In one embodiment, the invention is for use in infants, young children and/or children.

The nutritional composition can be administered (or given or fed) at an age and for a period that depends on the needs. The nutritional composition can be for example given immediately after birth of the infants. The composition of the invention can also be given during the first week of life of the infant, or during the first 2 weeks of life, or during the first 3 weeks of life, or during the first month of life, or during the first 2 months of life, or during the first 3 months of life, or during the first 4 months of life, or during the first 6 months of life, or during the first 8 months of life, or during the first 10 months of life, or during the first year of life, or during the first two years of life or even more. In some particularly advantageous embodiments of the invention, the nutritional composition is given (or administered) to an infant within the first 4, 6 or 12 months of birth of said infant. In some other embodiments, the nutritional composition of the invention is given few days (e.g. 1, 2, 3, 5, 10, 15, 20 . . . ) , or few weeks (e.g. 1, 2, 3, 4, 5, 6, 7, 8, 9, 10 . . . ) , or few months (e.g. 1, 2, 3, 4, 5, 6, 7, 8, 9, 10 . . . ) after birth. This may be especially the case when the infant is premature, but not necessarily.

In one embodiment the composition of the invention is given to the infant or young child as a supplementary composition to the mother's milk. In some embodiments the infant or young child receives the mother's milk during at least the first 2 weeks, first 1, 2, 4, or 6 months. In one embodiment the nutritional composition of the invention is given to the infant or young child after such period of mother's nutrition, or is given together with such period of mother's milk nutrition. In another embodiment the composition is given to the infant or young child as the sole or primary nutritional composition during at least one period of time, e.g. after the 1st, 2nd or 4th month of life, during at least 1, 2, 4 or 6 months.

### Combinations

In one embodiment, the mix of oligosaccharides consists of 2′FL and DFL.

In one embodiment of the invention, 2′FL is present in the combination or nutritional composition in an amount of 0.005-8 g/L of the combination or composition. In some embodiments, 2′FL may be in an amount of 0.01-3 g/L, such as 0.04-2 g/L or 0.05-1.5 g/L or 0.09-1.2 g/L. In a particular embodiment, 2′FL is in an amount of 1 g/L. In another particular embodiment, 2′FL is in an amount of 0.2 g/L.

2′FL can be present in the combination or nutritional composition in an amount of 0.004-6.8 g/100 g of the combination or composition on a dry weight basis, 2′FL may be present in an amount of 0.008-2.4 g/100 g, such as 0.03-1.6 g/100 g or 0.04-1.2 g/100 g or 0.07-1.0 g/100 g. In a particular embodiment, 2′FL is present in an amount of 0.8 g/100 g. In another particular embodiment, 2′FL is present in an amount of 0.16 g/100 g.

In another particular embodiment, 2′FL is in an amount of 5-500 g/L, 10 to 400 g/L, 40 to 300 g/L, 60-200 g/L, 80-180 g/L, 100-150 g/L or 110-130 g/L. In a particular embodiment, the 2′-FL is in an amount of 120 g/L. Such amounts are particularly adequate when the nutritional composition is in the form of a supplement or of a fortifier.

When the supplement or fortifier is in powder form 2′FL is preferably provided in the nutritional composition of the present invention in such an amount of 0.05-5 g, 0.1-4.5 g, 0.15-4 g, 0.2 to 3.5 g, 0.25 to 3, 0.3 to 2.5, 0.35 to 2, 0.4 to 1.5 g, 0.45-1 g, 0.5 to 0.75 g for example 0.6 g per serving.

In some embodiments, DFL is in an amount of 0.0005-0.5 g/L of the combination or composition, such as 0.001-0.3 g/L or 0.002-0.2 g/L or 0.01-0.25 g/L or 0.015-0.2 g/L or 0.025-0.19 g/L or 0.075-0.165 g/L of the combination or composition. In a particular embodiment, DFL is in an amount of 0.1 g/L. In another particular embodiment, DFL is in an amount of 0.025 or 0.026 g/L. In one embodiment DFL is in an amount of more than 0.01 g/L and optionally less than 0.1 g/L or more than 0.02 and less than 0.08 g/L. In one embodiment DFL is present in an amount of at least 0.01 g/L, at least 0.025, at least 0.026, at least 0.05, at least 0.07, at least 0.08, at least 0.1, at least 0.125, at least 0.15 at least 0.15 or at least 0.2 g/L.

In some embodiments, DFL is present in the combination or composition in a total amount of 0.0004-0.38 g/100 g of combination or composition on a dry weight basis. DFL may be in an amount of 0.0008-0.23 g/100 g, such as 0.0015-0.15 g/100 g, or 0.008-0.19 g/100 g or 0.012-0.15 g/100 g or 0.015-0.15 g/100 g or 0.019-0.15 g/100 g. In a particular embodiment, DFL is in an amount of 0.0075 or 0.078 g/100 g. In another particular embodiment, DFL is in an amount of 0.02 g/100 g. In a particular embodiment, DFL is in an amount of at least 0.001 g/100 g, at least 0.002 g/100 g, at least 0.005 g/100 g, at least 0.01 g/100 g, at least 0.02 g/100 g, at least 0.025 g/100 g, at least 0.04 g/100 g, at least 0.05 g/100 g, at least 0.075 g/100 g, at least 0.09 g/100 g, at least 0.1 g/100 g, at least 0.15 g/100 g, at least 0.2 g/100 g or at least 0.3 g/100 g.

In a particular embodiment, the DFL is provided in the combination or nutritional composition of the present invention in such an amount that normal consumption of the combination or nutritional composition would provide to the infant, young child, or child, consuming it a total daily dose of 0.003-3.9 g, preferably 0.006-3 g or 0.05-3 g or 0.1-3 g, for example 1.1-2.8 g per day.

In a particular embodiment, the DFL is provided in the combination or nutritional composition of the present invention in such an amount that one serving of the combination or nutritional composition would provide to the infant, young child, or child, consuming it a total dose of 0.003-3.9 g, preferably 0.006-3 g or 0.05-2.5 g or 0.1-2 g, for example 0.02 to 0.07 g per serving.

In a particular aspect of the invention, the combination or nutritional composition is an infant formula comprising an HMO mix consisting of 2′-FL and DFL wherein:
2′-FL is in an amount of 1-2 g/L, preferably 1.3 g/L, of the composition, and/or in an amount of 0.8-1.5 g/100 g, preferably 1 g/100 g of composition on a dry weight basis; and/or
DFL is in an amount of 0.1 to 0.3 g/L, preferably 0.2 g/L, of the composition and/or in an amount of 0.08-0.2 g/100 g, preferably 0.2 g/100 g of composition on a dry weight basis.

In another particular aspect of the invention, the combination or nutritional composition is a supplement or a fortifier and comprises an HMO mix consisting of 2′-FL and DFL wherein:
2′-FL is in an amount of 100-110 g/L, preferably 105 g/L, of the composition; and/or
DFL is in an amount of 10-20 g/L, preferably 15 g/L, of the composition.

In a particular aspect, 2′-FL and DFL are present in the HMO mix in a ratio 2′-FL: DFL of from 5:1 to 14:1, such as 5:1 to 12:1, 5:1 to 10:1, 6:1 to 10:1 or 8:1 to 10:1. In particularly advantageous embodiments, this ratio is 9:1 or around 9:1.

2′-FL and DFL may be isolated by chromatography or filtration technology from a natural source such as animal milks. Alternatively, the HMO may be produced by biotechnological means using specific fucosyltransferases and/or fucosidases either through the use of enzyme-based fermentation technology (recombinant or natural enzymes) or microbial fermentation technology. In the latter case, microbes may either express their natural enzymes and substrates or may be engineered to produce respective substrates and enzymes. Single microbial cultures and/or mixed cultures may be used. 2′-FL and DFL formation can be initiated by acceptor substrates starting from any degree of polymerization (DP), from DP=1 onwards. Alternatively, 2′-FL and DFL may be produced by chemical synthesis from lactose and free fucose. 2′-FL and DFL are also available commercially, for example from Glycom A/S in Denmark or Jennewein GmBH in Germany. 2′FL may be synthesised as described for example in “Large-scale synthesis of H-antigen oligosaccharides by expressing Helicobacter pylori alpha1,2-fucosyltransferase in metabolically engineered Escherichia coli cells” (Drouillard S, Driguez H, Samain E. Angew Chem Int Ed Engl. 2006 Mar. 3; 45(11): 1778-80) or be obtained from commercial sources.

In one embodiment of the present invention, the combination or nutritional composition comprises a mix of HMOs consisting of 2′FL and DFL. It is to be understood that when the expression “a mix of HMOs consisting of 2′FL and DFL” is used in the context of the present invention, such expression excludes the presence of HMOs different from 2′FL and DFL in the mix. In particular the mix is devoid of N-acetylated oligosaccharides, sialylated oligosaccharides, fucosylated oligosaccharides other than 2′-FL and DFL and precursors of HMOs such as sialic acid or fucose. In some embodiments, the combination or nutritional composition is devoid of N-acetylated oligosaccharides, sialylated oligosaccharides, fucosylated oligosaccharides other than 2′-FL and DFL and precursors of HMOs such as sialic acid or fucose.

In one embodiment, the mix of oligosaccharides consists of LNT and 6′SL.

In one embodiment of the invention, LNT is present in the combination or nutritional composition in an amount of 0.005-3 g/L of the composition. In some embodiments, LNT may be in an amount of 0.01-1.5 g/L of the composition, such as 0.04-1.2 g/L or 0.05-1 g/L or 0.09-0.8 g/L of the composition. In a particular embodiment, LNT is in an amount of 0.5 g/L of the composition. In another particular embodiment, LNT is in an amount of 0.1 g/L of the composition.

LNT can be present in the combination or nutritional composition in an amount of 0.004-2.3 g/100 g of composition on a dry weight basis, LNT may be present in an amount of 0.008-1.2 g/100 g of composition, such as 0.03-0.9 g/100 g or 0.04-0.8 g/100 g or 0.07-0.6 g/100 g of the composition. In a particular embodiment, LNT is present in an amount of 0.38 g/100 g of the composition. In another particular embodiment, LNT is present in an amount of 0.08 g/100 g of the composition.

In another embodiment of the invention the combination or nutritional composition may comprise from 0.005-5 g/L of 6′SL, or from 0.008-2.5 g/L, or from 0.01-1 g/L, or from 0.03-0.7 g/L, for example 0.04 or 0.5 g/L of LNT.

The nutritional composition according to the invention can contain 0.004-3.8 g of LNT per 100 g of composition on a dry weight basis, e.g. 0.006-1.9 g or 0.008-0.8 g or 0.023-0.5 g or 0.031-0.4 of LNT per 100 g of composition on a dry weight basis, for example 0.18 g or 0.04 g per 100 g of composition on a dry weight basis.

In a particular embodiment, the LNT is provided in the nutritional composition of the present invention in such an amount that normal consumption of the nutritional composition would provide to the infant or young child, respectively the child, consuming it a total daily dose of 0.003-3.9 g, preferably 0.006-2 g or 0.02-1.6 g or 0.03-1.3 g, for example 0.05-1 g per day.

LNT may be synthesised chemically by enzymatic transfer of saccharide units from donor moieties to acceptor moieties using glycosyltransferases as described for example in U.S. Pat. No. 5,288,637 and WO 96/10086.

6′SL formation can be initiated by acceptor substrates starting from any degree of polymerisation (DP), from DP=1 onwards. Alternatively, 6′SL may be produced by chemical synthesis from lactose and free N′-acetylneuraminic acid (sialic acid).

In one embodiment of the invention, 6′SL is present in the combination or nutritional composition in an amount of 0.005-5 g/L of the composition. In some embodiments, 6′SL may be in an amount of 0.08-2.5 g/L of the composition, such as 0.01-1 g/L or 0.03-0.07 g/L or 0.04-0.5 g/L of the composition. In a particular embodiment, 6′SL is in an amount of 0.24 g/L of the composition. In another particular embodiment, 6′SL is in an amount of 0.05 g/L of the composition.

6′SL can be present in the combination or nutritional composition in an amount of 0.004-3.8 g/100 g of composition on a dry weight basis, 6′SL may be present in an amount of 0.006-1.9 g/100 g of composition, such as 0.008-0.8 g/100 g or 0.23-0.5 g/100 g or 0.031-0.4 g/100 g of the composition. In a particular embodiment, 6′SL is present in an amount of 0.18 g/100 g of the composition. In another particular embodiment, 6′SL is present in an amount of 0.04 g/100 g of the composition.

In a particular aspect, the 6′SL and the LNT comprised in the composition or nutritional composition according to the invention are typically present in a ratio 6′SL: LNT of from 3:1 to 1:3, such as 2:1 to 1:2 or 2:1 to 1:1. In a particularly advantageous embodiment, this ratio is 2:1 or around 2.1 preferably this ratio is 1:1 or around 1:1.

In a particular aspect of the invention, the combination or nutritional composition comprises 6′SL and LNT wherein:
6′SL is in an amount of 0.005-5 g/L of the composition and/or in an amount of 0.004-3.8 g/100 g of composition on a dry weight basis; and/or
LNT is in an amount of 0.005-3 g/L of the composition and/or in an amount of 0.004-2.3 g/100 g of composition on a dry weight basis.

In a particular aspect of the invention, the combination or nutritional composition comprises 6′SL and LNT wherein:
6′SL is in an amount of 0.008-2.5 g/L of the composition and/or in an amount of 0.006-1 . 9 g/100 g of composition on a dry weight basis; and/or
LNT is in an amount of 0.01-1.5 g/L of the composition and/or in an amount of 0.008-1.2 g/100 g of composition on a dry weight basis.

In another particular embodiment the combination or nutritional composition of the present invention comprises 6′SL and LNT wherein:
6′SL is in an amount of 0.01-1 g/L of the composition and/or in an amount of 0.008-0.8 g/100 g of composition on a dry weight basis; and/or
LNT is in an amount of 0.04-1.2 g/L of the composition and/or in an amount of 0.03-0.9 g/100 g of composition on a dry weight basis.

In another particular embodiment the combination or nutritional composition of the present invention comprises 6′SL and LNT wherein:
6′SL is in an amount of 0.03-0.7 g/L of the composition and/or in a total amount of 0.023-0.5 g/100 g of composition on a dry weight basis; and/or
LNT is in an amount of 0.05-1 g/L of the composition and/or in an amount of 0.04-0.8 g/100 g of composition on a dry weight basis.

In another particular embodiment the combination or nutritional composition of the present invention comprises 6′SL and LNT wherein:
6′SL is in an amount of 0.04-0.5 g/L of the composition and/or in an amount of 0.031-0.4 g/100 g of composition on a dry weight basis; and/or
LNT is in an amount of 0.09-0.8 g/L of the composition and/or in an amount of 0.07-0.6 g/100 g of composition on a dry weight basis.

In a specific embodiment the combination or nutritional composition of the present invention comprises 6′SL and LNT wherein:
6′SL is in an amount of 0.24 or 0.05 g/L of the composition and/or in an amount of 0.18 or 0.04 g/100 g of composition on a dry weight basis; and/or
LNT is in an amount of 0.5 g/L or 0.1 g/L of the composition and/or in an amount of 0.38 g/100 g of composition or 0.08 g/100 g of composition on a dry weight basis.

In a particular embodiment, 6′SL is provided in the combination or nutritional composition of the present invention in such an amount that normal consumption of the nutritional composition or growing-up milk would provide to the infant, young child, or child, consuming it a total daily dose of 0.003 to 6.5 g, preferably 0.005-3.3 g or 0.006-1.3 g or 0.02-0.9 g, for example 0.024-0.7 g per day.

In a particular embodiment, the LNT is provided in the nutritional composition or growing-up milk of the present invention in such an amount that normal consumption of the nutritional composition or growing-up milk would provide to the infant, young child, or child, consuming it a total daily dose of 0.003-3.9 g, preferably 0.006-2 g or 0.02-1.6 g or 0.03-1.3 g, for example 0.05-1 g per day.

In one embodiment of the present invention, the combination or nutritional composition comprises a mix of HMOs consisting of LNT and 6′SL. It is to be understood that when the expression “a mix of HMOs consisting of LNT and 6′SL” is used in the context of the present invention, such expression excludes the presence of HMOs different from LNT and 6′SL in the mix. In particular the mix is devoid of N-acetylated oligosaccharides, sialylated oligosaccharides, fucosylated oligosaccharides other than LNT and 6′SL and precursors of HMOs such as sialic acid or fucose. In some embodiments, the combination or nutritional composition is devoid of N-acetylated oligosaccharides, sialylated oligosaccharides, fucosylated oligosaccharides other than LNT and 6′SL and precursors of HMOs such as sialic acid or fucose.

In a preferred embodiment of the invention, the HMO mix may be present in an amount of 0.15-15 g/L of the composition, such as 0.5-10 g/L or 0.75-7.5 g/L or 1-5 g/L or 1 to 2 g/L of the composition. In a particular embodiment, the HMO mix is in an amount of 1.5 g/L of the composition. In a preferred embodiment, the HMO mix is in an amount of 2 g/L of the composition. Such amounts are particularly adequate when the nutritional composition is in the form of a complete nutrition such as an infant formula, or in the case of a growing-up milk.

In case wherein the combination or nutritional composition is in powder form, the HMO mix may preferably be present in an amount of 0.11-11 g/100 g of composition on a dry weight basis, such as 0.4-7.5 g/100 g or 0.6-6 g/L or 0.8-3.8 g/100 g or 0.8-1.5 g/100 g of composition on a dry weight basis. In a particular embodiment, the HMO mix is in an amount of 1.1 g/100 of composition on a dry weight basis. Such amounts are particularly adequate when the combination or nutritional composition is in the form of a complete nutrition such as an infant formula, or in the case of a growing-up milk.

In another particular embodiment, the HMO mix is in an amount of 5-500 g/L, 10 to 400 g/L, 40 to 300 g/L, 60-200 g/L, 80-180 g/L, 100-150 g/L or 110-130 g/L of the composition. In a particular embodiment, the HMO mix is in an amount of 120 g/L. Such amounts are particularly adequate when the nutritional composition is in the form of a supplement or of a fortifier.

When the supplement or fortifier is in powder form the HMO mix is preferably provided in the nutritional composition of the present invention in such an amount of 0.05-5 g, 0.1-4.5 g, 0.15-4 g, 0.2 to 3.5 g, 0.25 to 3, 0.3 to 2.5, 0.35 to 2, 0.4 to 1.5 g, 0.45-1 g, 0.5 to 0.75 g for example 0.6 g per serving.

In one embodiment, the HMO mix is provided in the combination or nutritional composition of the present invention in such an amount that normal consumption of the nutritional composition would provide to the infant or young child, respectively the child, consuming it a total daily dose of 0.1 to 10 g, such as 0.2-9 g, 0.3-8 g, 0.4-7 g, 0.5-6 g, 0.6-5 g, 0,8-3 g, 0.9-2 g or 1 to 1.5 g per day.

When GOS, 2′FL and DFL are combined, all three oligosaccharides are considered responsible for the effect, e.g. for treating and/or preventing stress and/or a mood disorder.

When GOS, LNT and 6′SL are combined, all three oligosaccharides are considered responsible for the effect, e.g. for treating and/or preventing stress and/or a mood disorder.

The mix of HMOs as described herein is used in combination with GOS.

In a particular embodiment, the GOS is provided in the combination or nutritional composition of the present invention in such an amount that one serving of the combination or nutritional composition would provide to the infant, young child, or child, consuming it a total dose of 0.003-3.9 g, preferably 0.006-3 g, 0.05-2.5 g, 0.1-2 g or 0.2-1.5 g, for example 0.36 to 1.22 g per serving. Suitably, the GOS is provided in the combination or nutritional composition of the present invention in such an amount that one serving of the combination or nutritional composition would provide to the infant, young child, or child, consuming it a total dose of 0.001, 0.005, 0.01, 0.005, 0.1, 0.5, 1, 1.5, 2, 2.5, 3, 3.5 or 4 g per serving, or any range formed thereby.

### Other Ingredients

The nutritional composition according to the present invention may also comprise other types of oligosaccharide(s) (i.e. other than human milk oligosaccharides mentioned above) and/or a fiber(s) and/or a precursor(s) thereof. The other oligosaccharide and/or fiber and/or precursor thereof may be selected from the list comprising HMOs, fructo-oligosaccharides (FOS), inulin, xylooligosaccharides (XOS), polydextrose and any combination thereof. They may be in an amount between 0 and 10% by weight of composition. In a particular embodiment, the nutritional composition or the growing-up milk can also contain at least one BMO (bovine milk oligosaccharide).

Additional HMOs which may be included in the combination or nutritional composition according to the present invention may be selected from the group consisting of 3-FL (3-fucosyllactose), Lacto-difucotetraose (LDFT)), lacto-N-fucopentaose (e.g. lacto-N-fucopentaose I, lacto-N-fucopentaose II, lacto-N-fucopentaose III, lacto-N-fucopentaose V), lacto-N-fucohexaose, lacto-N-difucohexaose I, fucosyllacto-N-hexaose, fucosyllacto-N-neohexaose, difucosyllacto-N-hexaose I, difucosyllacto-N-neohexaose II, para-lacto-N-neohexaose (para-LNnH), LNnT (lacto-N-neotetraose), lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose, lacto-N-decaose, 3-SL (3′ sialyllactose) and any combination thereof.

In some embodiments, the combination or nutritional composition according to the invention comprises at least one additional HMO. In other embodiments, the combination or nutritional composition according to the present invention is devoid of any further HMOs.

The nutritional composition according to the invention may contain an additional protein source. Protein sources based on whey, casein and mixtures thereof may be used as well as protein sources based on soy (for example soy protein isolate from Gushen Biological Techn. Group Co. Ltd) and/or rice (for example HyprolRice Advance from Kerry of Ireland). As far as whey proteins are concerned, the additional protein source may be based on acid whey or sweet whey or mixtures thereof and may include alpha-lactalbumin and beta-lactoglobulin in any desired proportions. Suitably, the additional protein source is casein, skim milk, or whole milk. For example, a typical recipe may combine skim milk with the protein source as described herein above to reach the desired casein: whey ratio.

The nutritional composition according to the present invention generally contains a carbohydrate source. This is particularly preferable in the case where the nutritional composition of the invention is an infant formula. In this case, any carbohydrate source conventionally found in infant formulae such as lactose, sucrose, saccharose, maltodextrin, starch and mixtures thereof may be used although one of the preferred sources of carbohydrates is lactose.

The nutritional composition according to the present invention generally contains a source of lipids. This is particularly relevant if the nutritional composition of the invention is an infant formula. In this case, the lipid source may be any lipid or fat which is suitable for use in infant formulae. Some suitable fat sources include palm oil, structured triglyceride oil, high oleic sunflower oil and high oleic safflower oil, medium-chain-triglyceride oil. The essential fatty acids linoleic and a-linolenic acid may also be added, as well small amounts of oils containing high quantities of preformed arachidonic acid and docosahexaenoic acid such as fish oils or microbial oils. The fat source may have a ratio of n−6 to n−3 fatty acids of about 5:1 to about 15:1; for example about 8:1 to about 10:1.

The nutritional composition of the invention may also contain all vitamins and minerals understood to be essential in the daily diet and in nutritionally significant amounts. Minimum requirements have been established for certain vitamins and minerals. Examples of minerals, vitamins and other nutrients optionally present in the composition of the invention include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population.

If necessary, the nutritional composition of the invention may contain emulsifiers and stabilizers such as soy, lecithin, citric acid esters of mono-and di-glycerides, and the like.

The nutritional composition of the invention may also contain other substances which may have a beneficial effect such as lactoferrin, nucleotides, nucleosides, and the like. The nutritional composition of the invention may also contain carotenoid(s). In some particular embodiments of the invention, the nutritional composition of the invention does not comprise any carotenoid.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

Those skilled in the art will understand that they can freely combine all features of the present invention disclosed herein. In particular, features described for the product of the present invention may be combined with the method of the present invention and vice versa. Further, features described for different embodiments of the present invention may be combined. Where known equivalents exist to specific features, such equivalents are incorporated as if specifically referred to in this specification.

Further advantages and features of the present invention are apparent from the figures and non-limiting examples.

### EXAMPLES

The synthesis of melatonin by gut microbiota was measured using an in vitro digestion and fermentation model.

### In Vitro Digestion and Fermentation

A milk matrix was subjected to oral, gastric and small intestinal digestion procedures according to the standardized INFOGEST 2.0 method (Brodkorb A et al. Nature Protocols 2019; 14:991-1014). Then in vitro fermentation was achieved for 24 h in reactor containing:
10% of the digested milk matrix
80% of fermentation medium containing microbiota derived from fresh fecal samples, recovered from 3 healthy toddlers aged 3.2-3.3 years-old
10% of the test ingredients, consisting of
   Buffer only
   Galacto-oligosaccharides at the final concentration of 2 g/L
   Blend of 2′FL and DFL (ratio 9:1), at a final concentration of 2 g/L
   Blend of LNT and 6′SL (ratio 2:1), at a final concentration of 2 g/L
   A combination of (i) GOS and (ii) the blend of 2′FL and DFL (ratio 9:1), both at a final concentration of 2 g/L
   A combination of (i) GOS and (ii) the blend of LNT and 6′SL (ratio 2:1), both at a final concentration of 2 g/L Each combination was tested in triplicate. After 24 hours, fermentation medium was collected for metabolomics analysis.

### Metabolomics

The untargeted LC-MS/MS was carried out using a Thermo Scientific Vanquish LC coupled to Thermo Q Exactive HF MS. An electrospray ionization interface was used as ionization source. Analysis was performed in negative and positive ionization mode. The UPLC was performed using a slightly modified version of the protocol described by Doneanu et al. (Doneanu, C. E. UPLC/MS Monitoring of Water-Soluble Vitamin Bs in Cell Culture Media in Minutes. Waters application note (2011) https://www.waters.com/webassets/cms/library/docs/720004042en.pdf). Peak areas were extracted using Compound Discoverer 3.1 (Thermo Scientific). In addition to the automatic compound extraction by Compound Discoverer 3.1, a manual extraction of compounds included in an in-house library was performed using Skyline 21.1 (MacCoss Lab Software).

For each blend B, we calculate log2(Melatonin(B)/Melatonin(Milk)) that is, the base-2 logarithm of the ratio between the value of Melatonin for the blend and the value of Melatonin for milk.

### Statistics

Mixed linear models with random intercepts were used to assess the effect of the blends on the level of each metabolite.

The goodness of fit was assessed using the marginal and the conditional R2. The marginal R2 (marginal coefficient of determination) represents the variance explained by the fixed effects while the conditional R2 is interpreted as a variance explained by the entire model, including both fixed and random effects. In addition, we inspected the standardized residuals of each model to check whether outliers in the data were potentially of concern.

The ratio of the between-cluster variance to the total variance is called the Intraclass Correlation (ICC) was generally between 0.6 and 0.8. This can also be interpreted as the correlation among observations within the same cluster (donor).

P-values, approximated with the Kenward-Roger method, were used to determine which blends were significantly associated with the outcome.

### Results

Exposure of microbiome to digested milk matrix increased the abundance of melatonin, likely due to the presence of tryptophan, a precursor of melatonin. In addition to the milk matrix, the presence of either GOS or a blend of LNT and 6′SL did not trigger any effect on melatonin abundance. Conversely, in presence of a blend comprising 2′FL/DFL increased the synthesis of melatonin by the microbiota. Combination of GOS with HMOs significantly increased the amount of melatonin secreted, and this amount was higher upon exposure with GOS+2′FL/DFL than GOS+LNT/6′SL.

Altogether, the results indicate that the combination according to the present invention plays a role in promoting the biosynthesis of melatonin by the gut microbiota.

1. A method of promoting sleep and/or treating and/or preventing a sleep disorder in a subject, the method comprising administering a combination of galacto-oligosaccharides (GOS) and a mix of human milk oligosaccharides (HMOs) to the subject, wherein the mix of HMOs consists of 2′-fucosyllactose (2′FL) and difucosyllactose (DFL) or lacto-N-tetraose (LNT) and 6′-sialyllactose (6′SL).

2. The method of claim 1, wherein the combination is administered in a nutritional composition.

3. The method of claim 1, wherein the mix of HMOs consists of 2′-fucosyllactose (2′FL) and difucosyllactose (DFL).

4. The method according to claim 1, wherein the combination is administered in an amount that promotes sleep or treat and/or prevents sleep disorders by increasing production of melatonin by gut microbiota of the subject.

5. The method according to claim 2, wherein the nutritional composition does not comprise melatonin.

6. The method according to claim 1, wherein the sleep disorder is one or more of insomnia, poor sleep quality, sleep awaking, wakefulness, circadian rhythm disorders, parasomnias, narcolepsy, sleep-related movement disorders and sleep-related breathing disorders (SBDs).

7. The method according to claim 2, wherein the GOS are present in an amount of 2.4 g/L or 1.1 to 2.8 g/100 g of the nutritional composition on a dry basis.

8. The method according to claim 2, wherein the mix of HMOs consists of 2′FL and DFL.

9. The method according to claim 1, wherein the mix of HMOs consists of LNT and 6′SL.

10. The method composition according to claim 2, wherein:
(i) a) 2′FL is present in an amount of 0.005-8 g/L or 0.004-6.8 g/100 g of composition on a dry weight basis of the nutritional composition on a dry weight basis; and/or
b) DFL is present in an amount of 0.1 to 0.3 g/L or 0.08-0.2 g/100 g of the nutritional composition on a dry weight basis; or
(ii) a) LNT is present in an amount of 0.005-3 g/L or 0.004-2.3 g/100 g of the nutritional composition on a dry weight basis; and/or
b) 6′SL is present in an amount of 0.005 to 5 g/L or 0.004 to 3.8 g/100 g of the nutritional composition on a dry weight basis.

11. The method according to claim 2, wherein the nutritional composition further comprises a protein source, a carbohydrate source and a lipide source.

12. The method according to claim 2, wherein the nutritional composition is an infant formula, a starter infant formula, a follow-on or a follow-up infant formula, a growing up milk, a baby food, an infant cereal composition, a fortifier, a supplement, a beverage or a food.

13. The method according to claim 1, wherein the subject is an adult subject, an adolescent, an infant, a young child and/or a child.

14. (canceled)

15. A method for increasing production of melatonin by the gut of a subject, the method comprising administering to the subject a combination of galacto-oligosaccharides (GOS) and a mix of human milk oligosaccharides (HMOs), wherein the mix of HMOs consists of 2′-fucosyllactose (2′FL) and difucosyllactose (DFL) or lacto-N-tetraose (LNT) and 6′-sialyllactose (6′SL).

16. The method of claim 15, wherein the combination is administered in a nutritional composition.

17. The method according to claim 16, wherein the nutritional composition does not comprise melatonin.

18. The method according to claim 16, wherein the GOS are present in an amount of 2.4 g/L or 1.1 to 2.8 g/100 g of the nutritional composition on a dry basis.

19. The method according to claim 15, wherein the mix of HMOs consists of 2′FL and DFL.

20. The method according to claim 15, wherein the mix of HMOs consists of LNT and 6′SL.

21. The method according to claim 16, wherein:
(i) a) 2′FL is present in an amount of 0.005-8 g/L or 0.004-6.8 g/100 g of composition on a dry weight basis of the nutritional composition on a dry weight basis; and/or
b) DFL is present in an amount of 0.1 to 0.3 g/L or 0.08-0.2 g/100 g of the nutritional composition on a dry weight basis; or
(ii) a) LNT is present in an amount of 0.005-3 g/L or 0.004-2.3 g/100 g of the nutritional composition on a dry weight basis; and/or
b) 6′SL is present in an amount of 0.005 to 5 g/L or 0.004 to 3.8 g/100 g of the nutritional composition on a dry weight basis.
